(19) United States
(12) Patent Application Publication
(10) Pub. No.: US 2004/0193014 A1
(43) Pub. Date: Sep. 30, 2004

(54) Electric bending endoscope

(75) Inventors: Takayasu Miyagi, Tokyo (JP); Keiichi Arai, Tokyo (JP); Kouta Ishibiki, Tokyo (JP); Hiroki Moriyama, Tokyo (JP); Akira Suzuki, Yamanashi (JP); Toshinari Maeda, Tokyo (JP); Yuichi Ikeda, Tokyo (JP)

Correspondence Address: SCULLY SCOTT MURPHY & PRESSER, PC, 400 GARDEN CITY PLAZA, GARDEN CITY, NY, 11530

(73) Assignee: OLYMPUS OPTICAL CO., LTD., TOKYO (JP)

(21) Appl. No.: 10/397,996

(22) Filed: Mar. 26, 2003

(51) Int. Cl. A61B 1/005

(52) U.S. Cl. 600/146; 600/118

(57) ABSTRACT

An electric bending endoscope includes a bending driving portion which bends a bending portion arranged to a distal side of an inserting portion, a bending operation input portion which instructs and inputs a bending operation of the bending portion, and a preventing device which prevents the bending operation of the bending portion which is not intended by an operator.

### BACKGROUND OF THE INVENTION

1. Field of the Invention

The present invention relates to an electric bending endoscope which electrically bends a bending portion arranged to a distal side of an inserting portion.

2. Description of the Related Art

Recently, an endoscope is widely used. Various curing treatments are performed by inserting an elongated inserting portion in the celom so as to observe the organ in the celom or by using a treatment tool inserted in a processing tool channel as needed. In the industrial field, the endoscope observes and examines inner scratches and corrosion of a steam generator, a turbine, an engine and a chemical plant by inserting an elongated inserting portion.

The endoscope has a bending portion which is freely bent on a proximal side of a distal portion in the elongated inserting portion. Further, the endoscope inputs an instruction as the amount of bending, corresponding to a bending portion or a bending speed of the bending portion, by means for inputting a bending operation such as a bending operation lever or a joystick arranged to an operating portion. In the endoscope, a bending operation wire is mechanically stretched and contracted based on the amount of bending inputted as the instruction so as to bend the bending portion.

The above-mentioned endoscope includes an electric bending endoscope in which a built-in motor as bending driving means is controlled for rotation, that is, for normal rotation or reversal rotation, the motor's driving force enables the bending operation wire to stretch and contract, and the bending portion is electrically bent.

It is an object of the present invention to provide an electric bending endoscope which can prevent the bending operation which is not intended by an operator of the bending portion.

### SUMMARY OF THE INVENTION

According to the present invention, an electric bending endoscope comprises: a bending driving portion which bends a bending portion arranged to a distal side of an inserting portion; a bending operation input portion which instructs and inputs a bending operation of the bending portion; and a preventing device which prevents the bending operation of the bending portion which is not intended by an operator.

The above and other objects, features and advantages of the invention will become more clearly understood from the following description referring to the accompanying drawings.

### BRIEF DESCRIPTION OF THE DRAWINGS

FIG. 1 is a diagram showing the entire structure of an electric bending endoscope apparatus having an endoscope according to a first embodiment of the present invention;

FIG. 2 is a diagram schematically showing the electric bending endoscope apparatus shown in FIG. 1;

FIG. 3 is an explanatory diagram schematically showing a purpose detecting portion arranged to a grip portion in an operating portion;

FIG. 4 is a flowchart showing an example of bending control;

FIG. 5 is a diagram schematically showing an electric bending endoscope apparatus having an endoscope according to a second embodiment of the present invention;

FIG. 6 is a cross-sectional view schematically showing an example of the structure of a joystick having a validating switch;

FIGS. 7A and 7B are diagrams for explaining a bending operation input portion arranged to an electric bending endoscope according to a third embodiment of the present invention, FIG. 7A is a cross-sectional view schematically showing the structure of the bending operation input portion;

FIG. 7B is a plan view showing a ring-surface switch arranged to an inclining portion shown in FIG. 7A;

FIGS. 8A and 8B are diagrams for explaining a modification of the bending operation input portion shown in FIGS. 7A and 7B,

FIG. 8A is a cross-sectional view schematically showing the structure of the bending operation input portion;

FIG. 8B is a plane view showing a V-shaped groove formed in a bearing portion shown in FIG. 8A;

FIG. 9 is a cross-sectional view schematically showing the structure of the bending operation input portion arranged to an electric bending endoscope according to a fourth embodiment of the present invention;

FIGS. 10 to 17B are diagrams for explaining modifications of the bending operation input portion shown in FIG. 9,

FIG. 10 is a cross-sectional view schematically showing the structure of a bending operation input portion having a thick-formed rubber cover;

FIG. 11 is a cross-sectional view schematically showing the structure of the bending operation input portion using a track ball;

FIG. 12 is a cross-sectional view schematically showing the structure of the bending operation input portion using a magnet;

FIG. 13 is a cross-sectional view schematically showing the modification of the bending operation input portion shown in FIG. 12;

FIG. 14 is a cross-sectional view schematically showing the structure of a bending operation input portion having a plurality of ring pockets encapsulating viscous fluid;

FIGS. 15A and 15B are diagrams for explaining a modification of the bending operation input portion shown in FIG. 14,

FIG. 15A is an explanatory diagram schematically showing ring pockets modified in shape and arranged;

FIG. 15B is an explanatory diagram schematically showing the ring pockets encapsulating a changed type of the viscous fluid;

FIGS. 16A and 16B are diagrams for explaining the structure of the bending operation input portion using gears,

FIG. 16A is a cross-sectional view of the bending operation input portion in a single direction;

FIG. 16B is a perspective view schematically showing a main body frame shown in FIG. 16A;

FIGS. 17A and 17B are diagrams for explaining a modification of the bending operation input portion shown in FIGS. 16A and 16B,

FIG. 17A is a cross-sectional view schematically showing a driving wheel of the gears, formed by encapsulating the viscous fluid; and

FIG. 17B is a longitudinal cross-sectional view of FIG. 17A.

### DETAILED DESCRIPTION OF THE PREFERRED EMBODIMENTS

Hereinbelow, a description is given of embodiments of the present invention with reference to the drawings.

### First Embodiment

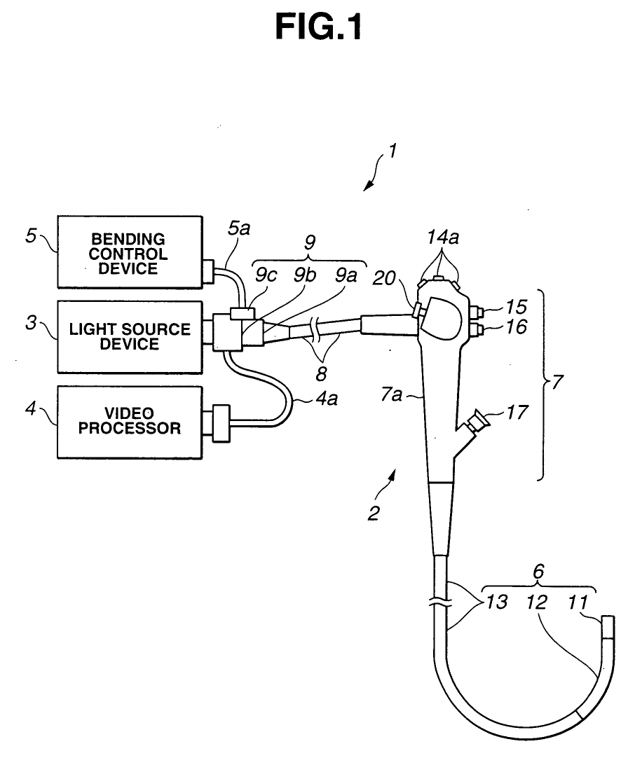

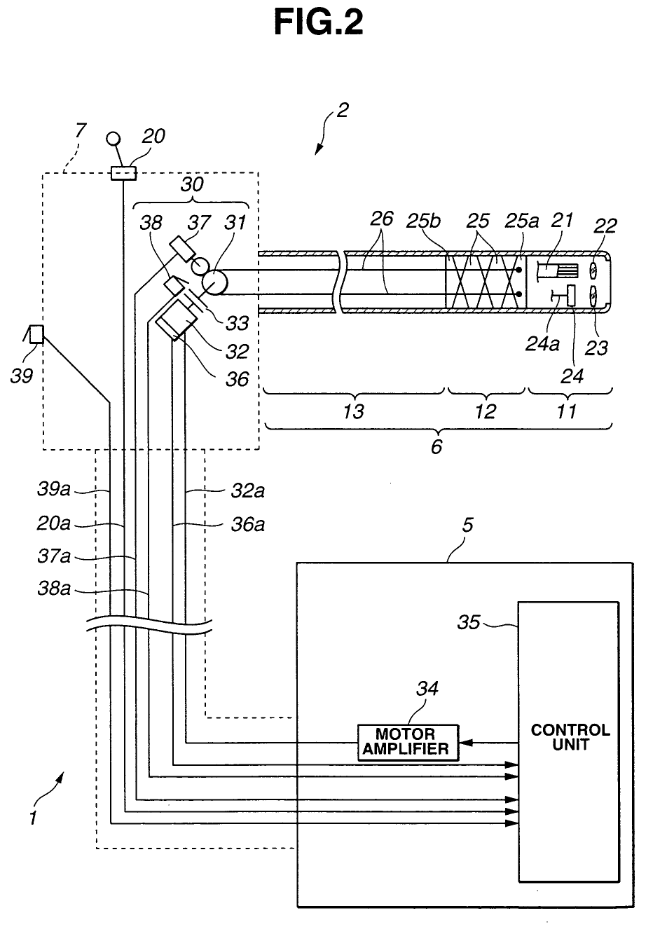

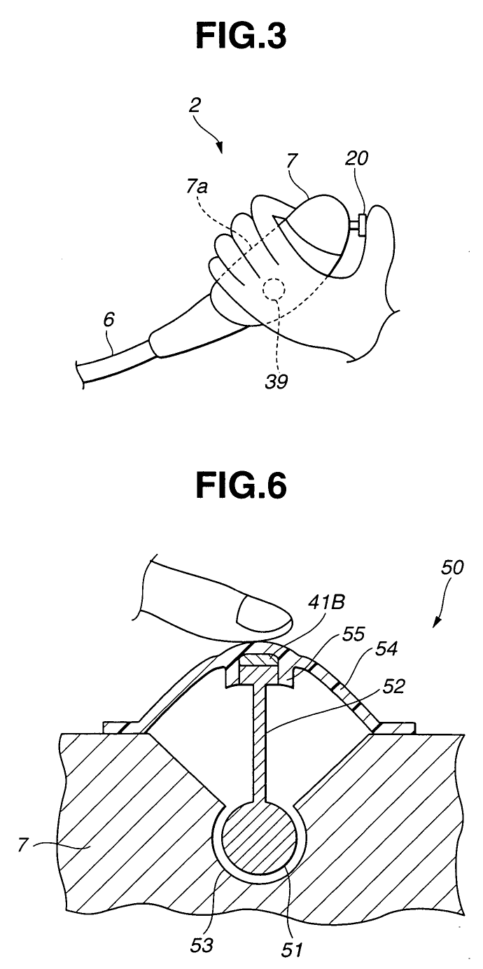

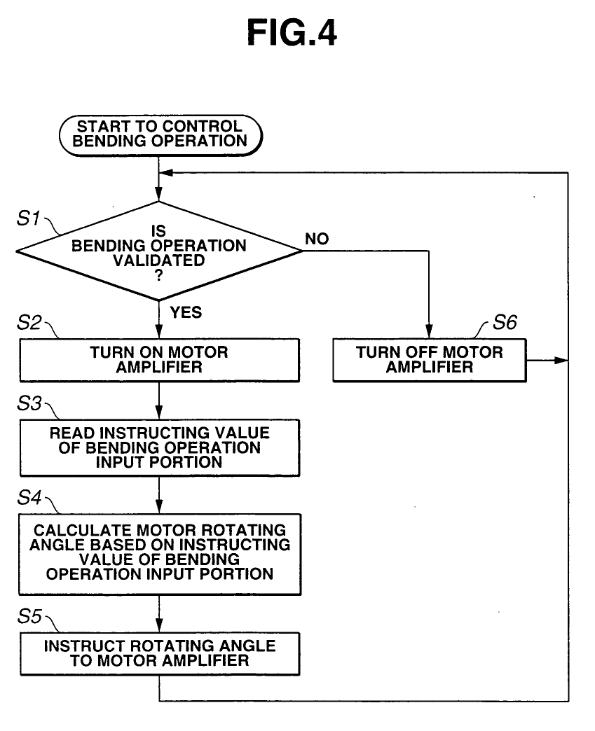

A first embodiment of the present invention will be described with reference to FIGS. 1 to 4. FIG. 1 is a diagram showing the entire structure of an electric bending apparatus having an endoscope according to the first embodiment of the present invention. FIG. 2 is a diagram schematically showing the structure of the electric bending endoscope apparatus shown in FIG. 1. FIG. 3 is an explanatory diagram showing a purpose detecting portion arranged to a grip portion in an operation portion. FIG. 4 is a flowchart showing an example of bending control.

Referring to FIG. 1, an electric bending endoscope apparatus 1 having an endoscope comprises an electric bending endoscope 2, a light source device 3, a video processor 4, and a bending control device 5 according to the first embodiment of the present invention. The electric bending endoscope 2 includes a bending driving portion (refer to FIG. 2) for electrically bending a bending portion, which will be described later, arranged to the distal side of an inserting portion. The light source device 3 supplies illumination light to the electric bending endoscope 2. The video processor 4 performs signal processing for image pick-up means incorporated in the electric bending endoscope 2, which will be described later. The bending control device 5 controls the driving operation of the bending driving portion in the electric bending endoscope 2. Incidentally, the video processor 4 is connected to a monitor (not shown), outputs a video signal to the monitor, and displays an endoscope image.

The electric bending endoscope 2 comprises an operating portion 7 which is continuously arranged to a proximal side of the inserting portion 6 and which commonly functions as a grip portion 7a. The electric bending endoscope 2 has a soft universal code 8 extending from a side portion at the operating portion 7. A light guide or a signal cable (which is not shown) is inserted in the universal code 8. A connector portion 9 is arranged to an end portion of the universal code 8. The connector portion 9 comprises a light guide connector (hereinafter, referred to as an LG connector) 9a detachably connected to the light source device 3 at the edge thereof, a video connector 9b to which a connecting cable 4a of the video processor 4 is detachably connected at a side portion of the LG connector 9a, and an angle connector 9c to which a connecting cable 5a of the bending control device 5 is detachably connected.

Continuously arranged to the endoscope inserting portion 6, that is, the inserting portion 6 of the electric bending endoscope 2 is a hard distal portion 11 arranged to the edge, a bending portion 12 which is arranged to a proximal side of the distal portion 11 and is freely bent, and a flexible tube portion 13 which is long and flexible and is arranged to a proximal side of the bending portion 12.

The endoscope operating portion 7, that is, the operating portion 7 of the electric bending endoscope 2 includes the grip portion 7a on the proximal side as a portion which is gripped by a user. In the endoscope operating portion 7, a plurality of video switches 14a for remote control of the video processor 4 are arranged at an upper portion of the grip portion 7a. Arranged to a side surface of the endoscope operating portion 7 are an air and water supply button 15 for operating the air and water supply operation and an suction button 16 for a suction operation.

Further, a processing tool inserting slit 17 for inserting a processing tool such as a biopsy clamp is arranged near a front end of the grip portion 7a of the endoscope operating portion 7. The processing tool inserting slit 17 is connected to a channel for inserting the processing tool (not shown). A processing tool such as a clamp (not shown) is inserted in the processing tool inserting slit 17, the edge of the processing tool is projected from the opening of the channel formed to the distal portion 11 via the channel for inserting the processing tool for biopsy, and the biopsy is performed.

The endoscope operating portion 7 comprises a bending operation input portion 20 for inputting the operation for bending the bending portion 12 such as a joystick or a track ball.

Referring to FIG. 2, a light guide 21 for transmitting the illumination light is inserted in the inserting portion 6 in the electric bending endoscope 2. The light guide 21 reaches the connector portion 9 of the universal code 8 via the operating portion 7 from the proximal side so as to transmit the illumination light from a light source lamp (not shown) arranged in the light source device 3. The illumination light transmitted from the light guide 21 illuminates a subject such as an affected part of the body from an edge surface of an illumination window (not shown) fixed to the distal portion 11 of the inserting portion via an illumination optical system 22.

An illuminated subject image, namely, a subject image is captured from an observation window (not shown) arranged adjacently to the illumination window. The captured subject image is picked up by an image pick-up device 24 such as a CCD (Charge-Coupled Device) via an objective optical system 23 and is photoelectrically converted into an image pick-up signal. The image pick-up signal is transmitted via a signal cable 24a extending from the image pick-up device 24, reaches the video connector 9b of the universal code 8 via the operating portion 7, and is outputted to the video processor 4 via the connecting cable 4a.

The video processor 4 processes the image pick-up signal from the image pick-up device 24 in the electric bending endoscope 2, generates a standard video signal, and displays the endoscope image on a monitor.

A bending piece 25a at the front end of rotatably connected bending pieces 25 forming the bending portion 12 is connected to a proximal portion of the distal portion 11 of the inserting portion in the electric bending endoscope 2. A bending piece 25b at the final end of the bending pieces 25 is connected to a distal side of the flexible portion 13.

A bending operation wire 26 is inserted in the inserting portion 2 to bend the bending portion 12 in the vertical and horizontal directions of an observation field of view. The edge of the bending operation wire 26 is fixed and held to the bending piece 25a at the front end by wax at positions corresponding to the vertical and horizontal directions of the bending portion 12. The bending portion 12 is bent in a desired direction and the distal portion 11 is positioned in a desired direction by stretching and contracting the bending operation wire 26 in accordance with the directions.

The bending operation wire 26 is stretched and contracted by the bending driving portion 30, thereby electrically bending the bending portion 12. Two bending operations wires 26 are shown in the vertical or horizontal direction in FIG. 2.

The bending driving portion 30 comprises a sprocket 31 which winds the proximal portion of the bending operation wire 26 for fixing and holding and stretches and contracts the bending operation wire 26, and a motor 32 which rotates the sprocket 31.

In the bending driving portion 30, a clutch 33 for disconnecting the driving force of the motor 32 is arranged between the sprocket 31 and the motor 32. Thus, the operation of the clutch 33 disconnects the transmission of the driving force of the motor 32 and the bending driving portion 30 enables a free operation of the angle. The clutch 33 is operated under the control of a control portion arranged to the bending control device 5, which will be described later. The clutch 33 may manually be operated.

A signal line 32a extending from the motor 32 is connected to an angle connector 9c of the universal code 8, and is further connected to the bending control device 5 via the connecting cable 5a so as to supply a motor driving signal to the motor 32 from a motor amplifier 34 arranged to the bending control device 5. The motor amplifier 34 is connected to the control portion 35 and is controlled by the control portion 35.

The motor 32 comprises an encoder 36 which detects a rotating position as means for detecting the rotating position. A signal line 36a extending from the encoder 36 is connected to the angle connector 9c of the universal code 8 and is further connected to the bending control device 5 via the connecting cable 5a. The encoder 36 outputs to the control portion 35, a detected rotating position signal indicating the rotating position of the motor 32.

The sprocket 31 converts the rotation of the motor 32 into advancing and returning motion of the bending operation wire 26. A potentiometer 37 for detecting the rotating position as the means for detecting the rotating position is connected to the sprocket 31. A signal line 37a extending from the potentiometer 37 is connected to the angle connector 9c of the universal code 8, and is further connected to the bending control device 5 via the connecting cable 5a. Thus, the potentiometer 37 outputs to the control portion 35, a rotating position signal indicating the rotating position of the sprocket 31.

Reference numeral 38 denotes a switch for detecting a clutch operation and detects whether the clutch 33 is ON or OFF. A signal line 38a extending from the switch 38 for detecting the clutch operation is connected to the angle connector 9c of the universal code 8, and is further connected to the bending control device 5 via the connecting cable 5a. Consequently, the clutch 33 outputs to the control portion 35, a clutch operation signal indicating the detected operation of the clutch 33.

As mentioned above, the bending operation input portion 20 such as the joystick or the track ball is arranged to the grip portion 7a of the operation portion 7 in the electric bending endoscope 2. The signal line 20a extending from the bending operation input portion 20 is connected to the angle connector 9c of the universal code 8, and is further connected to the bending control device 5 via the connecting cable 5. As a result, the bending operation input portion 20 outputs to the control device 35, a bending operation signal indicating the inputted bending operation.

The control portion 35 controls the motor amplifier 34 and drives the motor 32 in accordance with the bending operation signal from the bending operation input portion 20 based on the signals from the encoder 36 and the potentiometer 37 as the means for detecting the rotating position, thereby bending the bending portion 12.

When the operator erroneously operates and sharply moves the bending operation input portion 20 in the conventional electric bending endoscope, the bending portion 12 performs a sharp bending operation which does not correspond to the operator's purpose by driving the bending driving portion 30 in accordance with the bending operation signal of the bending operation input portion 20.

Then, according to the first embodiment, the electric bending endoscope 2 comprises a preventing device which prevents the bending operation of the bending portion 12 which is not intended by the operator.

According to the first embodiment, the electric bending endoscope 2 comprises a purpose detecting portion 39 which detects the purpose of the bending operation of the operator, thus to validate by the control portion 35 an input instruction of the bending operation input portion 20 based on the detected result of the purpose detecting portion 39.

The purpose detecting portion 39 is mounted on the operating portion 7. A signal line 39a extending from the purpose detecting portion 39 is connected to the angle connector 9c of the universal code 8, and is further connected to the bending control device 5 via the connecting cable 5a. Thus, the purpose detecting portion 39 outputs to the control portion 35, a detecting signal indicating the detected purpose of the bending operation of the operator.

Referring to FIG. 3, the purpose detecting portion 39 is formed on the grip portion 7a of the operating portion 7 in a shape to which the operator's hand touches. The purpose detecting portion 39 is, e.g., a pressure-sensitive sensor, a temperature sensor, or a vibration sensor which detects fine change in pressure, change in temperature, or vibration of the operator's hand upon gripping the grip portion 7a and operating the bending operation input portion 20.

The control device 35 controls the bending operation of the bending portion 12 based on the signal detected by the purpose detecting portion 39 in accordance with a flowchart shown in FIG. 4, which will be described later.

As mentioned above with reference to FIG. 1, the electric bending endoscope 2 with the foregoing structure is connected to the light source device 3, the video processor 4, and the bending control device 5 for the examination using the endoscope.

Referring to FIG. 3, the operator performs the examination using the endoscope by gripping the grip portion 7a in the electric bending endoscope 2 and by covering the purpose detecting portion 39 by the hand. During the examination using the endoscope, the operator bends the bending operation input portion 20 such as the joystick and bends the bending portion 20. In the electric bending endoscope 2, the bending portion 12 is bent as shown in the flowchart of FIG. 4.

In this case, the operator's thumb operates the bending operation input portion 20. Then, the purpose detecting portion 39 detects a fine motion of the operator's hand, detects the intention for the bending operation, and outputs a detecting signal to the control portion 35 in the bending control device 5.

The control portion 35 in the bending operation device 5 determines based on the detecting signal from the purpose detecting portion 39 whether or not the bending operation of the bending operation input portion 20 is validated (step S1).

When the bending operation is validated, the control portion 35 turns on the motor amplifier 34 (step S2). An instructing value (bending operation signal) as the input instruction of the bending operation input portion 20 is read (step S3).

The control portion 35 calculates a motor rotating angle based on the read instructing value (bending operation signal) of the bending operation input portion 20 (step S4). Further, the control portion 35 outputs the calculated value to the motor amplifier 34 and instructs the motor rotating angle (step S5). Then, the motor amplifier 34 drives the motor 32 at the instructed motor rotating angle.

The driving force of the motor 32 is transmitted to the sprocket 31 via the clutch 33, and the sprocket 31 rotates. Then, the bending operation wire 26 fixed and held to the sprocket 31 is stretched and contracted, so that the bending portion 12 performs a predetermined bending operation.

Until the operator bends the bending portion 12 again, the electric bending endoscope 2 maintains the above status.

For example, if the operator drops the electric bending endoscope 2 during the examination using the endoscope, the electric bending endoscope 2 falls down to the floor and the bending operation input portion 20 is sharply operated. In this case, since the operator does not grip the grip portion 7a in the electric bending endoscope 2, the operator's hand does not cover the purpose detecting portion 39.

Therefore, the purpose detecting portion 39 does not output the detecting signal and thus the control portion 35 in the bending control device 5 determines that the bending operation of the bending operation input portion 20 is invalidated, that is, NO in step S1 whereupon the motor amplifier 34 is turned off (step S6). Consequently, the bending operation of the bending portion 12 is not bent in the electric bending endoscope 2.

The electric bending endoscope 2 maintains the above status until the operator picks up the electric bending endoscope 2 from the floor, grips the grip portion 7a, and bends the bending portion 12 again.

As mentioned above, the bending operation of the bending portion 12 can be executed in the electric bending endoscope 2.

According to the first embodiment, the electric bending endoscope 2 prevents the bending operation of the bending portion 12 that is not intended by the operator.

According to the first embodiment, the electric bending endoscope 2 is applied to the electric endoscope which incorporates the image pick-up device 4 for picking up the captured subject image in the distal portion 11 of the inserting portion. However, the present invention is not limited to this and, of course, the electric bending endoscope may be applied to an optical endoscope having image transmitting means for transmitting the captured image, for enabling an eyepiece portion arranged to a rear end portion of the operating portion to observe the subject image transmitted by the image transmitting means.

Further, according to the first embodiment, the electric bending endoscope 2 is detachably connected to the bending control device 5, and the bending control device 5 controls the driving operation of the bending driving portion 30. However, the present invention is not limited to this and the electric bending endoscope 2 may include the bending control device 5.

### Second Embodiment

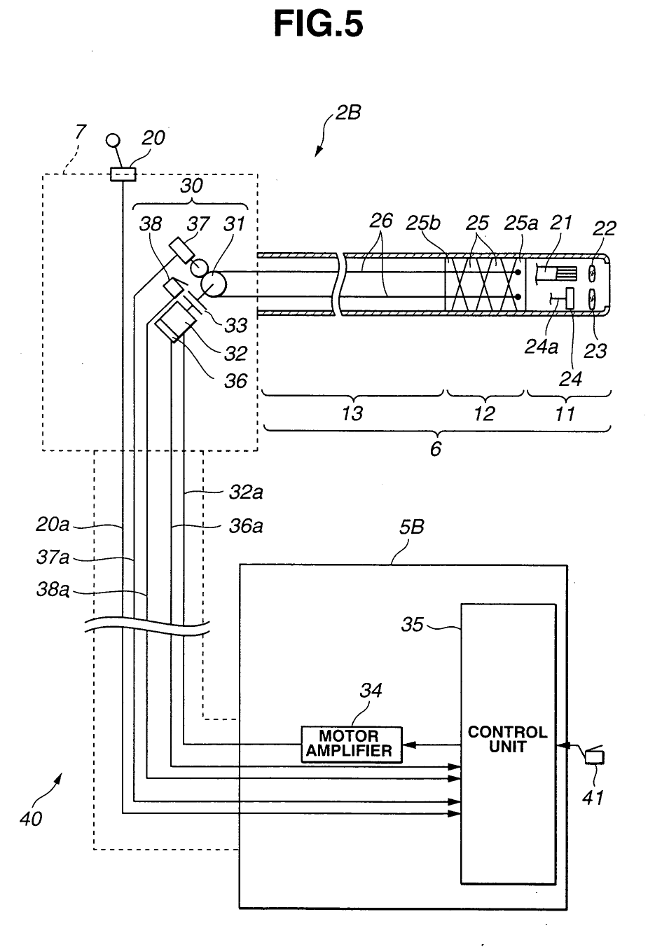

A description is given of a second embodiment of the present invention with reference to FIGS. 5 and 6. FIG. 5 is a diagram schematically showing an electric bending endoscope apparatus having an endoscope according to the second embodiment of the present invention. FIG. 6 is a cross-sectional view schematically showing an example of the structure of a joystick having a validating switch.

The endoscope according to the first embodiment comprises the purpose detecting portion 39 which detects the purpose for the bending operation of the operator as the preventing device for preventing the bending operation of the bending portion 12 that is not intended by the operator, and validates the input instruction of the bending operation input portion 20 based on the result detected by the purpose detecting portion 39. On the contrary, an endoscope according to the second embodiment comprises a validating switch which validates the input instruction of the bending operation input portion 20 as the preventing device for preventing the bending operation of the bending portion 12 that is not intended by the operator. Other structures are the same as those according to the first embodiment and therefore are not described, and the same reference numerals denote the same components.

Referring to FIG. 5, an electric bending endoscope apparatus 40 according to the second embodiment comprises a validating switch 41 which validates the input instruction of the bending operation portion 20 of an electric bending endoscope 2B in a bending control device 5B.

The validating switch 41 is connected to the control portion 35 in the bending control device 5B. For example, the validating switch 41 is a foot switch or a hand switch arranged at a standing position of the operator upon operating the endoscope. Incidentally, the validating switch 41 may be arranged to a front panel of the bending control device 5B.

The validating switch 41 outputs a validating signal to the control portion 35 of the bending control device 5B by a pressing operation. The control portion 35 of the bending control device 5B validates the bending operation of the bending operation input portion 20 and bends the bending portion 12 based on the validating signal from the validating switch 41.

The electric bending endoscope 2B with the above structure is connected to the light source device 3, the video processor 4, and the bending control device 5B for the examination using the endoscope, similarly to the first embodiment.

The operator performs the examination using the endoscope by gripping the grip portion 7a in the electric bending endoscope 2B. During the examination using the endoscope, the operator bends the bending portion 12 by the bending operation of the bending operation input portion 20 such as the joystick. In this case, the operator presses the validating switch 41 and the operates the bending operation input portion 20 by the thumb. Then, the validating switch 41 outputs the validating signal to the control portion 35 in the bending control device 5B.

The control portion 35 in the bending control device 5B validates the bending operation of the bending operation input portion 20 based on the validating signal from the validating switch 41, and bends the bending portion 12 in accordance with the bending operation of the bending operation input portion 20. In this case, the control portion 35 in the bending control device 5B performs the control operation in accordance with the flowchart similar to that shown in FIG. 4 described according to the first embodiment. The validating switch 41 may output the validating signal during the pressing operation or it may be an ON/OFF operating switch.

As a result, the same advantages as those according to the first embodiment are obtained in the electric bending endoscope 2B according to the second embodiment.

The validating switch 41 may be arranged to the joystick as the bending operation input portion 20 as shown in FIG. 6.

In a joystick 50 shown in FIG. 6, a stick portion 52 extends to a ball base portion 51. The ball base portion 51 is rotatably inserted and held to a bearing portion 53 formed to the operating portion 7.

An upper portion of the stick portion 52 is covered with a rubber cover 54 in the joystick 50. The rubber cover 54 comprises by a fixing portion 55 for holding and fixing a top portion of the stick portion 52 in the center portion on an inner peripheral side opposed to the joystick 50 and is fixed by adhesion to an outer surface of the operating portion 7 at an end portion thereof so as to prevent the leaking of water.

The joystick 50 is rotatably operated near the top portion of the rubber cover 54 so that the thumb presses it, thereby freely rotating the ball base portion 51 together with the stick portion 52.

Although not shown, the ball base portion 51 in the joystick 50 is connected to a potentiometer as the means for detecting the rotation position. The potentiometer is connected to the control portion 35 of the bending control device 5B and outputs a rotating position signal indicating a rotating position of the joystick 50.

According to a modification of the second invention, the joystick 50 comprises a validating switch 41B between the top portion of the stick portion 52 and the rubber cover 54. The validating switch 41B is turned on by pressing the top portion of the rubber cover 54, and outputs the validating signal to the control portion 35 of the bending control device 5B via a signal line (not shown). The validating switch 41B may be a switch for outputting the validating signal during the pressing operation or an ON/OFF operating switch.

Consequently, according to the modification, the validating switch 41B is used similarly to the validating switch 41 according to the second embodiment and the same advantages as those according to the second embodiment are obtained.

### Third Embodiment

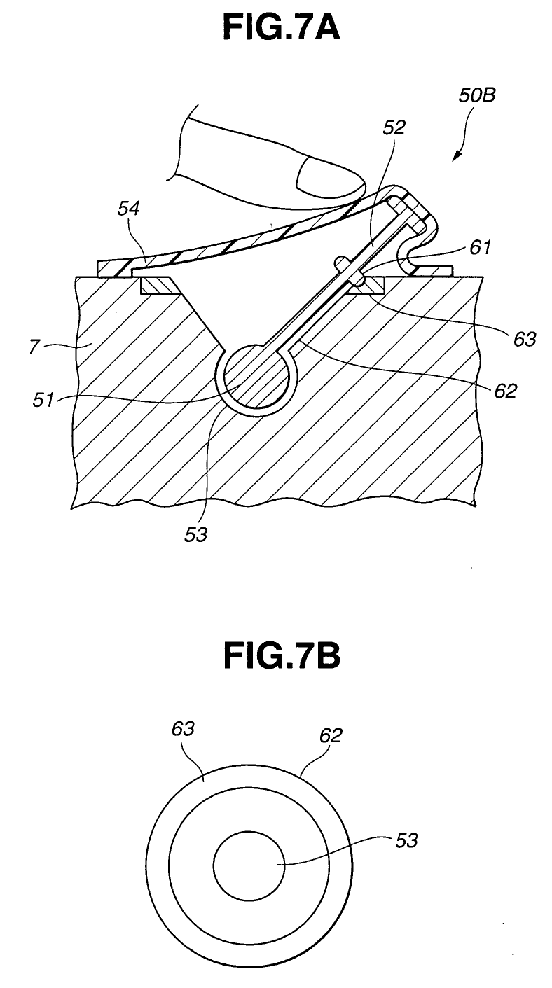

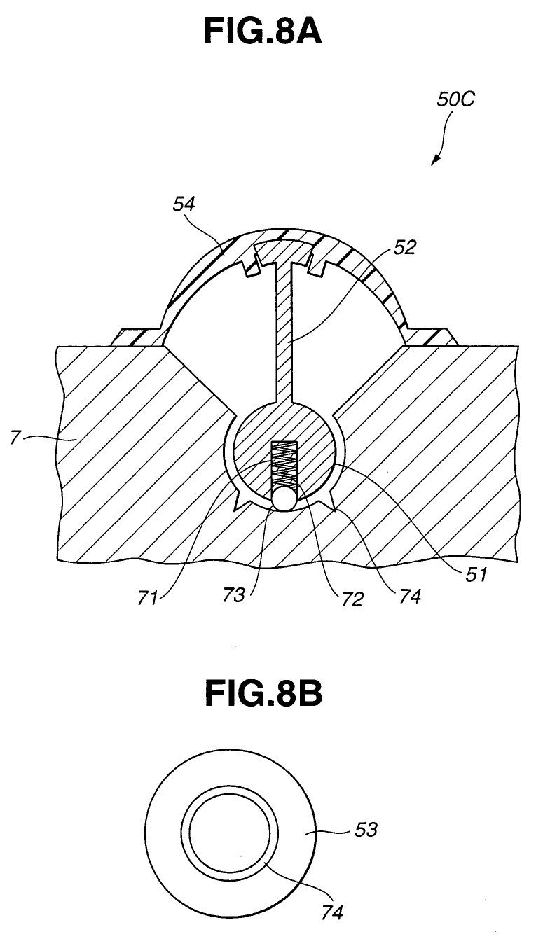

A description is given of a third embodiment of the present invention with reference to FIGS. 7A to 8B. FIGS. 7A and 7B are diagrams for explaining a bending operation input portion arranged to an electric bending endoscope according to the third embodiment of the present invention, FIG. 7A is a cross-sectional view schematically showing the structure of the bending operation input portion, and FIG. 7B is a plan view showing a ring-surface switch arranged to an inclining portion shown in FIG. 7A. FIGS. 8A and 8B are diagrams for explaining a modification of the bending operation input portion shown in FIGS. 7A and 7B, FIG. 8A is a cross-sectional view schematically showing the structure of the bending operation input portion, and FIG. 8B is a plan view showing a V-shaped groove formed to a bearing portion shown in FIG. 8A.

An electric bending endoscope according to the third embodiment comprises, as the preventing device for preventing the bending operation of the bending portion 12 that is not intended by the operator, a notifying device which notifies that the operating position of the bending operation input portion 20 matches a predetermined position upon the bending operation of the bending operation input portion 20.

Referring to FIG. 7A, in the electric bending endoscope according to the third embodiment, a joystick 50B as the bending operation input portion 20 includes, as the notifying device, a flange portion 61 projected to a predetermined portion of the stick portion 52 and a ring-surface switch 63 at a predetermined position of an inclining portion 62 of the operating portion 7.

Referring to FIG. 7B, the ring-surface switch 63 is concentrically arranged to a surface of the inclining portion 62. The ring-surface switch 63 may separately be arranged.

In the joystick 50B, when the stick portion 52 is operated and reaches the predetermined position, the flange portion 61 comes into contact with the ring-surface switch 63, thereby sending the sense like the click operation to the operator's thumb and notifying that the operating position reaches the predetermined position. At the operating position of the stick portion 52, for example, the bending angle of the bending portion 12 upon reversing the direction of the distal portion 11 of the inserting portion is 90° or more.

Further, the flange portion 61 comes into contact with the ring-surface switch 63, thereby turning on the ring-surface switch 63. Upon the ON operation of the ring-surface switch 63, it outputs to the control portion 35 of the bending control device 5B via a signal line (not shown), a signal for instructing the bending speed including an instruction for the bending direction in accordance with the contact position of the switch 63 at the constant bending speed, in place of outputting the signal for instructing the position which is outputted as a signal indicating the inclining position of the joystick 50B.

The control portion 35 of the bending control device 5B bends the bending portion 12 based on a speed instructing signal from the ring-surface switch 63. In this case, the bending speed is preset. The bending speed may be set in a plurality of steps.

The electric bending endoscope with the above structure is connected to the light source device 3, the video processor 4, and the bending control device 5 for the examination using the endoscope, similarly to the first embodiment.

The operator performs the examination using the endoscope by gripping the grip portion 7a in the electric bending endoscope. During the examination using the endoscope, the operator bends the bending portion 12 by bending the joystick 50B. In this case, the operator presses the joystick 50B by his thumb. In the joystick 50B, when the operating position reaches a reversing one at which the bending angle of the bending portion 12 is 90° or more, the flange portion 61 comes into contact with the ring-surface switch 63, thereby sending the sense like the click operation to the operator. As a consequence, the operator recognizes that the joystick 50B is operated at the position for reversing the bending portion 12.

Further, the flange portion 61 comes into contact with the ring-surface switch 63, thus to turn on the ring-surface switch 63. The ring-surface switch 63 outputs a signal for instructing the bending speed including the instruction for the bending direction corresponding to the contact position thereof at a constant bending speed, in place of outputting the position instruction as a signal of an inclining position of the joystick 50 to the control portion 35 of the bending control device 5B.

As a result, the same advantages as those according to the first and second embodiments are obtained according to the third embodiment. In addition, it is notified that the joystick 50B is operated to the position for reversing the bending portion 12 and the bending speed of the bending portion 12 after the reversing position can be set to be sufficiently slow.

The notifying device may be structured in the joystick as shown in FIGS. 8A and 8B.

Referring to FIG. 8A, a joystick 50C comprises a spring 72 fixed to a cave portion 71 at one end thereof. The cave portion 71 is formed to the ball base portion 51. A ball sliding portion 73 which is slid to a surface of the bearing portion 53 is fixed to another end of the spring 72 opposed to the surface of the bearing portion 53 in the operating portion 7, against energizing force of the spring 72. The notifying device is formed with a V-shaped groove 74 in which the ball sliding portion 73 is freely inserted at a predetermined position of the surface of the bearing portion 53. Referring to FIG. 8B, the V-shaped groove 74 is concentrically formed to the surface of the bearing portion 53.

When the stick portion 52 is operated and reaches the predetermined position, the ball sliding portion 73 is slid in accordance with the operation of the stick portion 52 and is inserted in the V-shaped groove 74, and the joystick 50C transmits the sense like the click operation to the operator's thumb, thus to notify that the operating position reaches the predetermined position.

Hence, the same advantages as those according to the third embodiment are obtained according to the modification.

### Fourth Embodiment

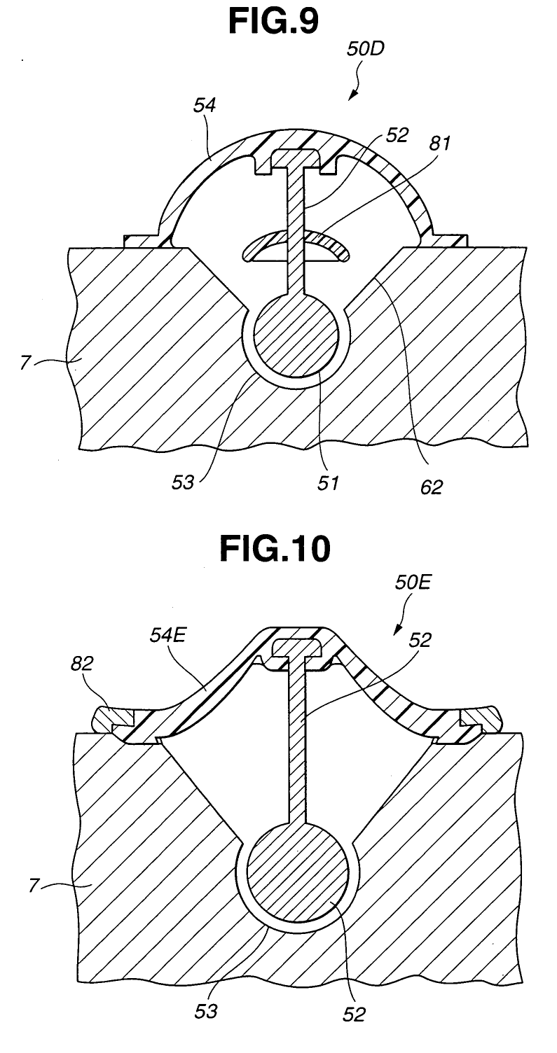

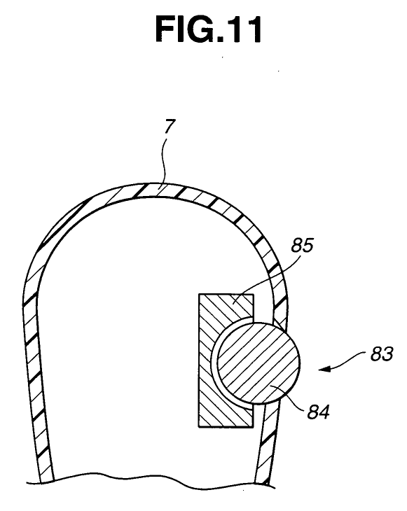

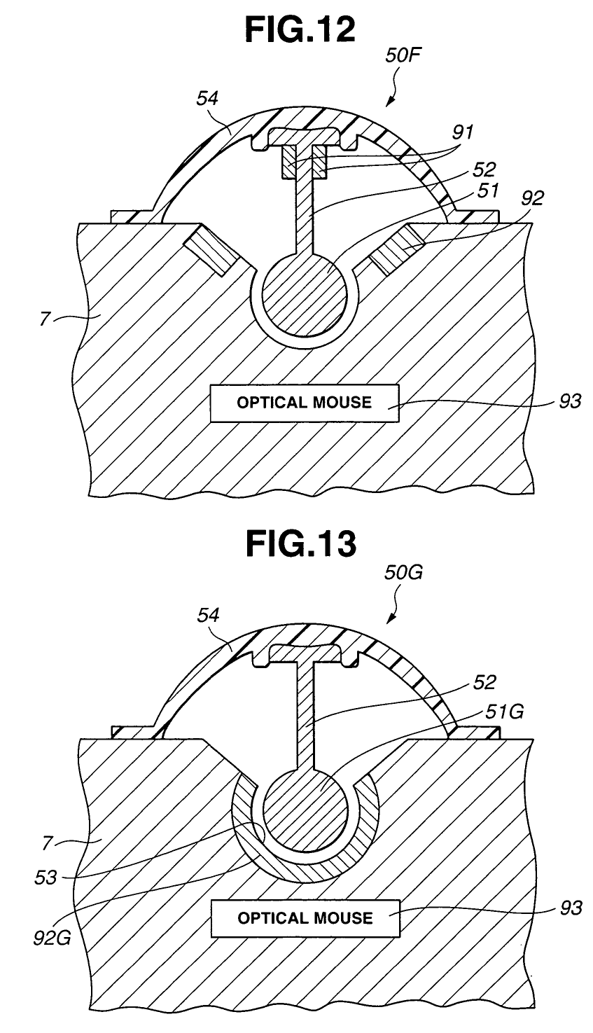

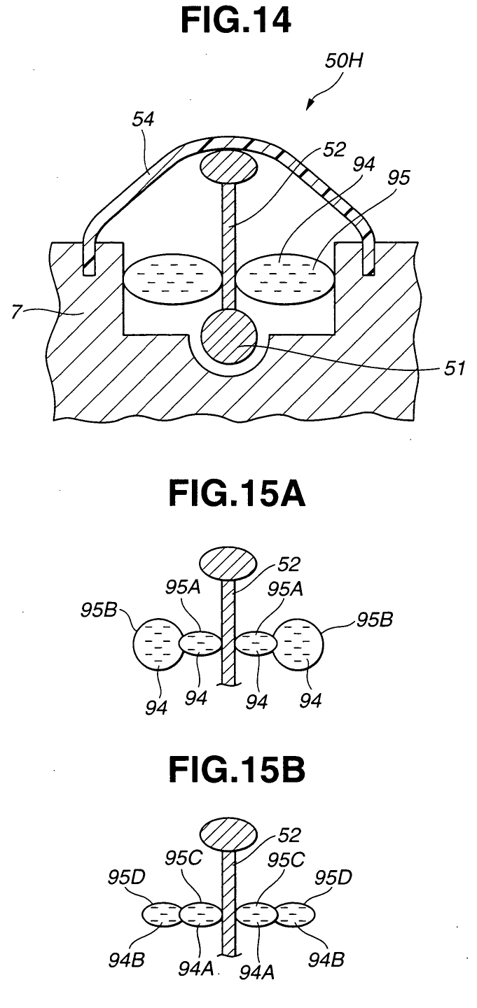

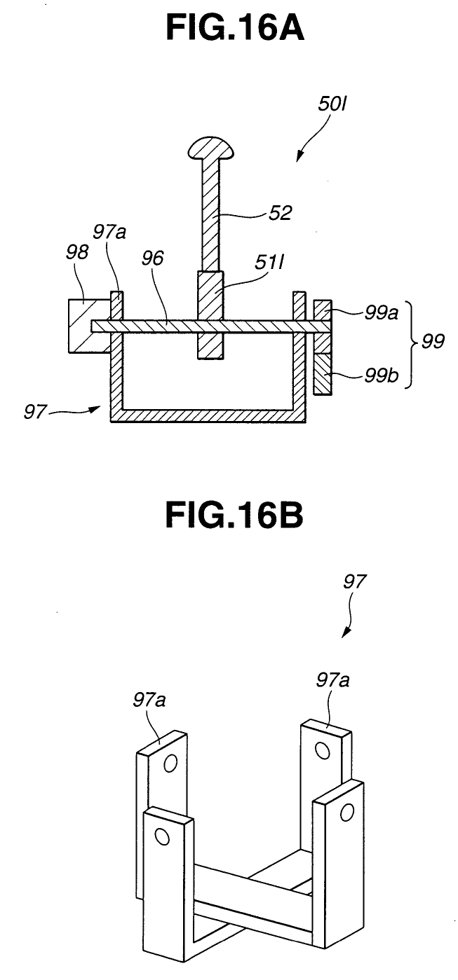

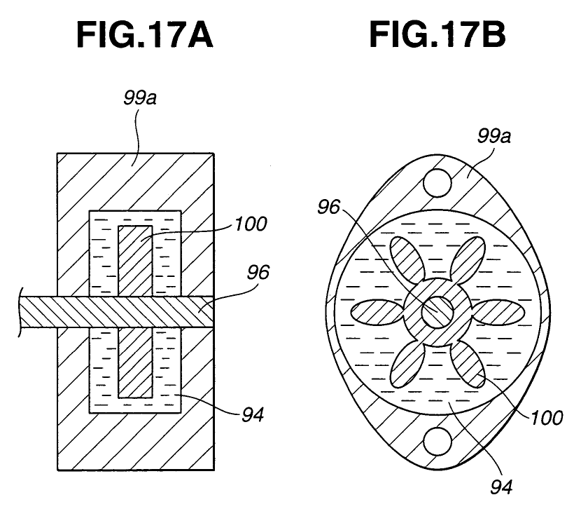

A description is given of a fourth embodiment of the present invention with reference to FIGS. 9 to 17B. FIG. 9 is a cross-sectional view schematically showing the structure of a bending operation input portion arranged to an electric bending endoscope according to the fourth embodiment of the present invention. FIGS. 10 to 17A are diagrams for explaining modifications of the bending operation input portion shown in FIG. 9. FIG. 10 is a cross-sectional view schematically showing the structure of a bending operation input portion having a thick-formed rubber cover. FIG. 11 is a cross-sectional view schematically showing the structure of the bending operation input portion using a track ball. FIG. 12 is a cross-sectional view schematically showing the structure of the bending operation input portion using a magnet. FIG. 13 is a cross-sectional view schematically showing the modification of the bending operation input portion shown in FIG. 12. FIG. 14 is a cross-sectional view schematically showing the structure of be bending operation input portion having a plurality of ring pockets encapsulating viscous fluid. FIGS. 15A and 15B are diagrams for explaining the modification of the bending operation input portion shown in FIG. 14, FIG. 15A is an explanatory diagram schematically showing ring pockets modified in shape and arranged, and FIG. 15B is an explanatory diagram schematically showing the ring pockets encapsulating a changed type of the viscous fluid. FIGS. 16A and 16B are diagrams for explaining the structure of the bending operation input portion using gears, FIG. 16A is a cross-sectional view of the bending operation input portion in a single direction, and FIG. 16B is a perspective view schematically showing a main body frame shown in FIG. 16A. FIGS. 17A and 17B are diagrams for explaining the modification of the bending operation input portion shown in FIGS. 16A and 16B, FIG. 17A is a cross-sectional view schematically showing a driving wheel of the gears, formed by encapsulating the viscous fluid, and FIG. 17B is a longitudinal cross-sectional view of FIG. 17A.

According to the fourth embodiment, as the preventing device which prevents the bending operation of the bending portion 12 that is not intended by the operator, an operating resistance device is provided to cause operating resistance in the bending operation input portion upon the bending operation of the bending operation input portion 20.

Referring to FIG. 9, an electric bending endoscope according to the fourth embodiment comprises a joystick 50D as the bending operation input portion 20 having a skirt elastic member 81 at a predetermined portion of the stick portion 52 as the operating resistance device.

Thus, when the stick portion 52 is operated and reaches beyond the predetermined position, the elastic member 81 comes into contact with a surface of the inclining portion 62, and the joystick 50D thus generates the operating resistance in the operator's thumb.

The operating resistance device will be structured in the joystick 50D as shown in FIG. 10.

Referring to FIG. 10, the operating resistance device is structured by making a rubber cover 54E thicker along a peripheral portion fixed to the surface of the operating portion 7 from the center to which the stick portion 52 is fixed. Reference numeral 82 denotes a fixing member which reinforces the rubber cover 54E.

Thus, the stick portion 52 is operated against elastic force caused by deformation of the rubber cover 54E and the joystick 50E causes the operating resistance in the thumb.

In the electric bending endoscope, the operating resistance device may be provided for the track ball as the bending operation input portion 20.

Referring to FIG. 11, in a track ball 83, a rotating ball 84 as an operating member is rotatably inserted and held to a ball bearing portion 85 arranged in the operating portion 7. The ball bearing portion 85 is made of a friction member, as the operating resistance device, which causes friction on a surface to which the rotating ball 84 is inserted and held. The ball bearing portion 85 is fixed and held by fixing means (not shown) in the operating portion 7.

Accordingly, the track ball 83 is rotated against the friction resistance of the friction member, thereby causing the operating resistance in the thumb.

Although not shown, in the track ball 83, the ball bearing portion 85 is connected to the potentiometer similarly to the joystick and the potentiometer outputs a rotating position signal to the control portion 35 of the bending control device 5B.

The operating resistance device may use magnetic force in the bending operation input portion 20.

Referring to FIG. 12, in a joystick 5OF as the bending operation input portion 20, an operating-side magnet 91 is provided at a predetermined portion of the stick portion 52 as the operating resistance device and a receiving-side magnet 92 is provided to react to the operating-side magnet 91 at a predetermined position of the inclining portion 62.

Consequently, the joystick 50F is operated against the reaction between the operating side-magnet 91 and the receiving-side magnet 92, thereby causing the operating resistance in the thumb.

The operating resistance device uses the magnetic force in the joystick 50F. As the rotating position detecting means, an optical mouse 93 is used. The optical mouse 93 is a photo-reflector which detects a Newton ring obtained by illuminating light to the ball base portion 51, thereby detecting the rotating position of the ball base portion 51.

In the joystick, the operating resistance device using the magnetic force may utilize not the reaction of the magnetic force but attracting force therebetween as shown in FIG. 13.

Referring to FIG. 13, in a joystick 50G, the ball base portion 51G as the operating resistance device is made of a magnetic member and a receiving-side magnet 92G is arranged to a predetermined portion of the surface of the bearing portion 53.

The joystick 50G is operated against the attracting force between the receiving-side magnet 92G and the ball base portion 51G, and thus causes the operating resistance in the thumb.

Upon using the magnetic force in the operating resistance device as mentioned above, the potentiometer may be utilized as the rotating position detecting means.

In the joystick, the operating resistance device may be made of ring pockets encapsulating viscous fluid such as gel as shown in FIG. 14.

Referring to FIG. 14, in a joystick 50H, the operating resistance device includes a plurality of ring pockets 95 encapsulating viscous fluid 94 such as gel to cover a predetermined portion of the stick portion 52.

Thus, the stick portion 52 is operated against viscosity of the viscous fluid 94 encapsulated in the pockets 95 in accordance with the deformation of the plurality of ring pockets 95, so that the joystick 50H causes the operating resistance in the thumb of the hand.

The joystick 50H may include the plurality of ring pockets 95 as shown in FIGS. 15A and 15B.

For example, referring to FIG. 15A, flat pockets 95A formed by modifying the shapes of the plurality of ring pockets 95 are arranged on the side of the stick portion 52 and spherical pockets 95B are arranged to the outer periphery of the flat pockets 95A in the joystick 50H.

Further, referring to FIG. 15B, in the joystick 50H, pockets 95C formed by changing the kind of the viscous fluid 94 encapsulated in the plurality of ring pockets 95 are arranged on the side of the stick portion 52 and viscous fluid 94A with low viscosity is encapsulated in the pockets 95C, and viscous fluid 94B with high viscosity is encapsulated in pockets 95D arranged to the outer periphery of the pockets 95C.

Referring to FIGS. 15A and 15B, in the joystick 50H, the plurality of ring pockets 95 are arranged. Thus, the operating resistance is reduced by decreasingly operating the stick portion 52 upon bending the bending portion 12 at a small bending angle and the operating resistance is increased by increasingly operating the stick portion 52 upon bending the bending portion 12 at a large bending angle.

In the joystick, the operating resistance device may use a gear as shown in FIGS. 16A and 16B. That is, the gear as the operating resistance device is arranged in the bending operation input portion and increases the operating resistance by the rotation of the gear.

Referring to FIGS. 16A and 16B, in a joystick 50I, two orthogonal rotating axes 96 are fixed to a base portion 51I from which the stick portion 52 extends and they are rotatably fixed to a main body frame 97 formed by orthogonally combining horseshoe frames 97a. FIG. 16A is a cross-sectional view schematically showing the joystick 50I from a single direction for the purpose of the brief description with the drawing.

In the joystick 50I, one end of the rotating axis 96 is connected to a potentiometer 98 as the rotating position detecting means arranged to the outside of the main body frame 97 thereby detecting the rotating position.

Further, in the joystick 50I, the operating resistance device is structured by connecting another end of the rotating axis 96 to a driving wheel 99a of a gear 99 arranged to the outside of the main body frame 97. Reference numeral 99b denotes a driven wheel 99b which is engaged to the driving wheel 99a.

As a consequence, the stick portion 52 is operated against the gears 99, so that the joystick 50I causes the operating resistance in the thumb of the hand.

Referring to FIGS. 17A and 17B, the viscous fluid 94 is encapsulated in the driving wheel 99a of the gears 99 and a wing portion 100 fixed to the rotating axis 96 may be arranged. That is, the operating resistance device uses the resistance of the fluid provided in the bending operating input portion. The wind portion 100 is rotated against the viscosity of the viscous fluid 94, thereby rotating the driving wheel 99a of the gears 99.

Thus, the stick portion 52 is operated against the viscosity of the viscous fluid 94, so that the joystick 50I causes the operating resistance in the thumb of the hand.

According to the fourth embodiment, the operating resistance is caused in the bending operation input portion and the endoscope may have a notifying device which notifies that the operating position of the bending operation input portion reaches a predetermined position similarly to the third embodiment.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. A electric bending endoscope comprising:
a bending driving portion which bends a bending portion arranged to a distal side of an inserting portion;
a bending operation input portion which instructs and inputs a bending operation of the bending portion; and
a preventing device which prevents the bending operation of the bending portion which is not intended by an operator.

2. An electric bending endoscope according to claim 1, wherein the preventing device comprises a purpose detecting portion which detects the purpose for the bending operation of the operator, and validates or invalidates the input instruction of the bending operation input portion based on a result detected by the purpose detecting portion.

3. An electric bending endoscope according to claim 2, wherein the purpose detecting portion is arranged to an operating portion which is connected to a proximal side of the inserting portion.

4. An electric bending endoscope according to claim 3, wherein the purpose detecting portion has a shape in which the hand of the operator comes into contact with a grip portion arranged to the operating portion.

5. An electric bending endoscope according to claim 3, wherein the purpose detecting portion is a sensor which detects whether or not the hand of the operator comes into contact with the operating portion.

6. An electric bending endoscope according to claim 2, wherein the purpose detecting portion is arranged to the bending operation input portion.

7. An electric bending endoscope according to claim 2, wherein the purpose detecting portion is arranged to a standing position of the operator.

8. An electric bending endoscope according to claim 1, wherein the preventing device is a notifying device which notifies that an operating position of the bending operation input portion reaches a predetermined position upon the bending operation of the bending operation input portion.

9. An electric bending endoscope according to claim 8, wherein the notifying device is arranged to an operating portion which is connected to a proximal portion of the inserting portion.

10. An electric bending endoscope according to claim 8, wherein the notifying device is arranged to the bending operation input portion.

11. An electric bending endoscope according to claim 10, wherein the notifying device transmits the sense like a click operation to the hand of the operator.

12. An electric bending endoscope according to claim 1, wherein the preventing device is an operating resistance device which causes operating resistance in the bending operation input portion upon bending the bending operation input portion.

13. An electric bending endoscope according to claim 12, wherein the operating resistance device includes a pocket member encapsulating viscous fluid set in the bending operation input portion.

14. An electric bending endoscope according to claim 12, wherein the operating resistance device uses friction resistance set in the bending operation input portion.

15. An electric bending endoscope according to claim 12, wherein the operating resistance device uses magnetic force set in the bending operation input portion.

16. An electric bending endoscope according to claim 12, wherein the operating resistance device increases the operating resistance as a bending angle is increased in accordance with the bending operation of the bending operation input portion.

17. An electric bending endoscope according to claim 12, wherein the operating resistance device uses means for increasing the operating resistance by rotation, arranged in the bending operation input portion.

18. An electric bending endoscope according to claim 12, wherein the preventing device has a notifying device which notifies that an operating position of the bending operation input portion reaches a predetermined position upon the bending operation of the bending operation input portion against the operating resistance of the operating resistance device.

19. An electric bending endoscope according to claim 18, wherein the notifying device causes the sense like a click operation in the hand of the operator.

20. An electric bending endoscope according to claim 12, wherein the operating resistance device uses fluid resistance set in the bending operation input portion.
